(19) United States
(12) Patent Application Publication
(10) Pub. No.: US 2024/0321401 A1
(43) Pub. Date: Sep. 26, 2024

(54) ACCELERATING PARTICLE SIMULATIONS USING MACHINE LEARNING AND MOLECULAR DYNAMIC SIMULATIONS

(71) Applicant: NEC Laboratories Europe GmbH, Heidelberg (DE)

(72) Inventors: Makoto Takamoto, Heidelberg (DE); Henrik Christiansen, Heidelberg (DE); Francesco Alesiani, Heidelberg (DE)

(21) Appl. No.: 18/324,199

(22) Filed: May 26, 2023

Related U.S. Application Data
(60) Provisional application No. 63/453,788, filed on Mar. 22, 2023.

(51) Int. Cl. G16C 10/00

(52) U.S. Cl. CPC G16C 10/00

(57) ABSTRACT

A method for performing a molecular dynamics simulation includes inputting an initial condition into an evolution model to predict a first condition at a next time step and inputting the initial condition into a molecular dynamics model to predict a second condition at the next time step. It is determined whether to use the first condition or the second condition as a prediction in the molecular dynamics simulation based on an estimated uncertainty associated with the evolution model.

### CROSS-REFERENCE TO PRIOR APPLICATION

Priority is claimed to U.S. Patent Application No. 63/453,788, filed on Mar. 22, 2023, the entire disclosure of which is hereby incorporated by reference herein.

### FIELD

The present invention relates to artificial intelligence (AI) and machine learning, and in particular, a method, system and computer-readable medium for accelerating particle simulations using machine learning and molecular dynamic simulations.

### BACKGROUND

Molecular dynamics simulation is one of the popular methods to study biological and chemical processes in industrial and academic communities. Conventional molecular dynamic simulation demands a high powered computing environment which prohibits many researchers from utilizing it.

### SUMMARY

In an embodiment, the present invention provides a method for performing a molecular dynamics simulation. The method includes inputting an initial condition into an evolution model to predict a first condition at a next time step and inputting the initial condition into a molecular dynamics model to predict a second condition at the next time step. It is determined whether to use the first condition or the second condition as a prediction in the molecular dynamics simulation based on an estimated uncertainty associated with the evolution model.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a framework for training of networks to accelerate particle simulations, according to an embodiment of the present invention;

FIG. 2 schematically illustrates a framework for performing inference using the trained networks, according to an embodiment of the present invention;

FIG. 3 is a block diagram of an exemplary processing system, which can be configured to perform any and all operations disclosed herein; and

FIG. 4 illustrates an exemplary flowchart for accelerating particle simulations according to an embodiment of the present invention.

### DETAILED DESCRIPTION

Embodiments of the present invention provide a method to accelerate the molecular dynamics simulations with the help of machine learning models that estimate the temporal evolution of the particles, the uncertainties of the estimations, and the evolution of the system. The machine learning prediction of temporal evolution allows faster calculation of the system evolution, and the uncertainties are used to control the calculation control pipe-line, and determine where and when to use either machine learning models or molecular dynamics simulation. In view of the uncertainties, the method also provides users with explanations of the method. Thus, the present invention improves existing approaches by providing for an explainable AI system. The combination of machine learning and molecular dynamics is more accurate than simply using molecular dynamics in generating particle simulations. Using a hybrid of machine learning and molecular dynamics for particle simulation is also especially advantageous as it not only significantly optimizes the speed of particle simulation with the resources required for particle simulation, but also accelerates the process. The method, in addition to providing higher accuracy, also saves computational resources, increases computing power and/or frees up computational resources for other tasks. Moreover, embodiments of the present invention can also be applied to effect improvements in the technical field of molecular dynamics, allowing for greater flexibility, more accurate simulations, as well as for discovery of more drugs, as biologists and chemists will be able to simulate interactions between particles much more easily.

Embodiments of the present invention can be especially advantageously applied not only for the optimization of computation resources, but also to improve the accuracy to the molecular dynamics simulations and the machine learning model.

The molecular dynamics simulation is one of the popular methods to study biological and chemical processes in both industrial and academic communities. However, molecular dynamics simulation in general demands a huge numerical cost which prohibits many researchers without high-performance computing environment to utilize it.

To solve this problem, the interest on applying machine learning methods to molecular dynamics simulation has recently been growing as an alternative method. Owing to the ability to emulate physics of a molecular dynamics system, machine learning models can avoid iterative calculation and limitation of the time-step size that are necessary for the molecular dynamics simulation.

However, present machine learning approaches are limited only to provide temporal evolution prediction (see Bapst, Victor, et al. “Unveiling the predictive power of static structure in glassy systems,” Nature Physics 16.4: 448-454 (2020), which is hereby incorporated by reference herein), or model prediction uncertainty (see Zuo, Yunxing, et al. “Performance and cost assessment of machine learning interatomic potentials,” The Journal of Physical Chemistry A 124.4: 731-745 (2020), which is incorporated by reference herein) and suffer from:
1. inability to take into account the uncertainty resulting from physical or chemical reaction, and
2. inability to optimize the switching between a molecular dynamics simulation and a machine learning prediction.

Embodiments of the present invention provide a hybrid method to address these issues based on a combination of machine learning models estimating system uncertainty using physical or chemical knowledge.

Traditional numerical methods (molecular dynamics simulations) for the simulation of drug discovery simulate the evolution of the system by numerically solving the PDEs modelling the system. On the other hand, it is possible for the deep neural networks to learn the behavior of the simulator, which can be used as a fast and efficient surrogate model of the numerical simulator. However, the former demands a huge numerical cost because of very small time-step size (˜1 femto-second) for numerical accuracy. Although the latter methods alleviate this small time-step problem, the accumulation of prediction error is much larger than the former method, which leads to a completely wrong prediction when the interaction is complex and strong.

Embodiments of the present invention provide a hybrid-approach with uncertainty estimation. The proposed hybrid method performs temporal update of the system with a machine learning model with large time-step size when the prediction uncertainty is below a threshold, and switches to the traditional molecular dynamics method when the prediction uncertainty becomes large. The prediction uncertainty includes: (1) model uncertainty, (2) global uncertainty. The model uncertainty is the intrinsic uncertainty in the machine learning models (also known as epistemic uncertainty). The global uncertainty captures the global information, such as indications of molecular structure changes. With those uncertainty measures, the hybrid method evolves the molecular dynamics system safely and effectively. Moreover, the hybrid method can flexibly be combined with any existing machine learning models and switches the main machine learning model to the new state-of-the-art model.

Embodiments of the present invention consider a system composed of a global model, an evolution model, a simulation updater, an uncertainty judge block, and a particle trajectory update block that work together to perform the molecular dynamics simulations using machine learning:
1. Global model: This model accepts either all the particle data or global geometric information, and estimates the uncertainty of global-geometrical changes. The uncertainty becomes large when global geometry may be rapidly changed within a few time-step future, for example.
2. Evolution model: This model accepts the coordinate and velocity (X^{k}) information of a particle and surrounding ones, and estimates either (coordinate, velocity) at the next timestep or the force value on those particles.
3. Simulation updater: This module accepts the coordinate and velocity (X^{k}) information of a particle and surrounding ones, and calculates (coordinate, velocity) at the next timestep of those particles using molecular dynamics/molecular Monte Carlo simulation's procedure.
4. Uncertainty judge block: This module accepts the estimated uncertainties by global and evolution models, and judges which updating information to be used.
5. Particle trajectory update block: This module accepts the decision of the uncertainty judge block, and performs temporal update using the prediction by either evolution model or Simulation updater.

The evolution and global model parameters are updated using the stochastic gradient descent as follows:

θ′=θ−∇_{θ}L(X_{MD}, X_{EV}; U_{EV}, U_{G}) where L is a loss function, such as mean-square-error and mean absolute error. Here the uncertainty value U_{G} and U_{EV} can also be penalized if uncertainty label is available.

According to a first aspect, the present disclosure provides a method for performing a molecular dynamics simulation. The method comprises inputting an initial condition into an evolution model to predict a first condition at a next time step, inputting the initial condition into a molecular dynamics model to predict a second condition at the next time step, and determining whether to use the first condition or the second condition as a prediction in the molecular dynamics simulation based on an estimated uncertainty associated with the evolution model.

According to a second aspect, the method according to the first aspect further comprises that the initial condition includes coordinate and velocity information of a particle and corresponding surrounding particles at an initial time step, and the first and second condition each include a prediction of a position of the particle at a next time step.

According to a third aspect, the method according to the first aspect or the second aspect further comprises that the estimated uncertainty associated with the evolution model is based on a global uncertainty.

According to a fourth aspect, the method according to any of the first to the third aspect further comprises that the global uncertainty is associated with a global model, and wherein the global model accepts particle information and computes the global uncertainty based on a change in a global geometry of the particle.

According to a fifth aspect, the method according to any of the first to the fourth aspect further comprises that the global uncertainty and the uncertainty associated with the evolution model are input to an uncertainty model, and wherein the uncertainty model performs an evaluation of the global uncertainty and the uncertainty associated with the evolution model by comparing to an uncertainty threshold.

According to a sixth aspect, the method according to any of the first to the fifth aspect further comprises that the determination of whether to use the first condition or the second condition is based on the evaluation by the uncertainty model, and wherein the first condition is used as the prediction in the molecular dynamics simulation based on a combination of the global uncertainty and the uncertainty associated with the evolution model being lower than the uncertainty threshold.

According to a seventh aspect, the method according to any of the first to the sixth aspect further comprises that the uncertainty model produces a Boolean value as a result of the evaluation.

According to an eighth aspect, the method according to any of the first to the seventh aspect further comprises that the global model is a trained neural network, and wherein the global model is trained to estimate an uncertainty of global-geometrical changes.

According to a ninth aspect, the method according to any of the first to the eighth aspect further comprises that a global uncertainty estimation rule is calculated for the global model, and wherein the global uncertainty estimation rule is based on a distance calculated between the particle and a target particle in the global model or a local potential energy of the particle.

According to a tenth aspect, the method according to any of the first to the ninth aspect further comprises that the determining whether to use the first condition or the second prediction as the prediction in the molecular dynamics simulation is based on the distance between the particle and the target particle or the local potential energy of the particle being either greater than or less than a target threshold.

According to an eleventh aspect, the method according to any of the first to the tenth aspect further comprises that the target threshold is calculated as a numerical multiple of a size of the target particle.

According to a twelfth aspect, the method according to any of the first to the eleventh aspect further comprises that the molecular dynamics simulation is a Monte Carlo simulation.

According to a thirteenth aspect, the method according to any of the first to the twelfth aspect further comprises that the evolution model is a trained neural network, and wherein the evolution model is trained using experimental and/or simulation data including the prediction used in the molecular dynamics simulation.

A fourteenth aspect of the present disclosure provides a computer system programmed for performing a molecular dynamics simulation comprising one or more hardware processors which, which, alone or in combination, are configured to provide for execution of the following steps: inputting an initial condition into an evolution model to predict a first condition at a next time step; inputting the initial condition into a molecular dynamics model to predict a second condition at the next time step; and determining whether to use the first condition or the second condition as a prediction in the molecular dynamics simulation based on an estimated uncertainty associated with the evolution model.

A fifteenth aspect of the present disclosure provides a tangible, non-transitory computer-readable medium having instructions thereon, which, upon being executed by one or more processors, provides for execution of the following steps: inputting an initial condition into an evolution model to predict a first condition at a next time step; inputting the initial condition into a molecular dynamics model to predict a second condition at the next time step; and determining whether to use the first condition or the second condition as a prediction in the molecular dynamics simulation based on an estimated uncertainty associated with the evolution model.

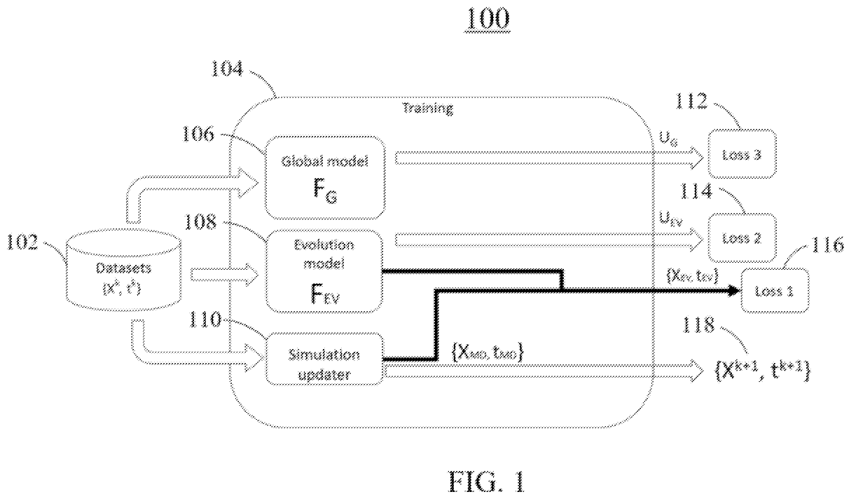

FIG. 1 schematically illustrates a framework for training of networks to accelerate particle simulations, according to an embodiment of the present invention. In FIG. 1, datasets 102 include all particle data. For example, the particle data includes current position, and velocity associated with each particle. The particle data can also include chemical and physical information associated with each particle. The chemical and physical properties include a charge on the particle, electronegativity of the particle, and a species with which the particle is associated. In some embodiments, the particle data includes coordinate and velocity information (X^{k}) of particles at corresponding time steps (t^{K}). The data from datasets 106 is provided to a global model (F_{G}) 106, an evolution model (F_{EV}) 108, and a simulation updater 110.

The global model 106 uses all the particle data or the global-geometrical changes to estimate an uncertainty of global-geometrical changes (U_{G}) 112. In some examples, the global-geometrical changes indicate structural changes of a protein molecule which could be measured by calculating the volume or surface area of the protein molecule. In some cases, the global uncertainty may be determined using historical particle data that is collected over a predetermined period of time. In some other cases, the global uncertainty may be determined using particle data is currently stored in the dataset 102. In some embodiments, the estimated uncertainty U_{G} 112 may become large when global geometry may rapidly change within a few time-steps. For example, a change involving more than a certain threshold of (e.g., 10%) of global geometry (e.g., surface area of a protein), within a certain threshold of time (e.g., 1%) in comparison with the total physical time in a simulation, can be considered a rapid change in a few time steps. In some embodiments, the global model 106 describes physical and chemical changes of a considering system. In such embodiments, the uncertainty U_{G} 112 of the global model 106 may be estimated from the observational data, such as temporal evolution of the drug and protein molecules. For example, the uncertainty U_{G} 112 may be estimated by comparing the predicted movement and interaction of drug and protein molecules with actual movement and interaction of drug and protein molecules to determine a difference between the actual movement and interaction and predicted movement and interaction to estimate the uncertainty U_{G} 112. In the case of the interaction between a drug and a protein, the distance measured between them can be used for the uncertainty measure because the interaction becomes strong as the distance decreases. For example, the global model 106 analyzes all the particle data or the global-geometric changes to determine a rate of change for the global-geometric changes or the particle data. In cases where the rate of change is over a threshold, the estimated uncertainty may be considered large.

The evolution model 108 accepts the coordinate and velocity (X^{k}) information of a particle and corresponding surrounding particles as input, and estimates either (coordinate, velocity) at the next timestep {X_{EV}, t_{EV}} or the force value on those particles using a machine learning model. For example, the machine learning model may be represented by a neural network (f), that takes the particle information (e.g., coordinate and velocity) as input, and produces an estimate of particle information (e.g., coordinate and velocity) at the next time step as output. This is represented using the following equation:

$${X}_{EV} , {t}_{EV} = f ( {X}^{k} , {t}^{k} )$$

Exemplary input data for the neural network function (f) includes coordinate information related to a particle, velocity information related to a particle, charge associated with a particle, and species associated with the particle. Exemplary output data of the neural network function (f) includes a prediction of particle information X_{EV} (e.g., coordinate, velocity) at the next time step (t_{EV}) and prediction uncertainty U_{EV}. In some embodiments, the uncertainty U_{EV} associated with the machine learning model of the evolution model 108 is calculated as uncertainty U_{EV} 114. In some other embodiments, the uncertainty U_{EV} may be measured as a standard deviation of the neural network's prediction, compared to the mean value of the neural network's prediction.

In some embodiments, the uncertainty U_{EV} 114 of the evolution model can be obtained using Bayesian method. One simple way is to construct the evolution model 108 using the families of probabilistic models, such as the Gaussian process and variational autoencoder (see Pinheiro Cinelli, Lucas; et al. “Variational Autoencoder,” Variational Methods for Machine Learning with Applications to Deep Networks, Springer, pp. 111-149 (2021), which is hereby incorporated by reference herein).

The simulation updater 110 accepts the coordinate and velocity (X) information of a particle and corresponding surrounding particles as input. The coordinate and velocity (X^{k}) information is used to calculate particle information (coordinate, velocity) at a next timestep {X_{MD}, t_{MD}} of the particle using molecular dynamics/molecular Monte Carlo simulation's procedure. In some embodiments, the molecular dynamics simulation uses the Newton equations to update particle trajectories. For example, the Newton equations include

$$\frac{dx}{dt} = v , and$$

$$m \frac{dv}{dt} = F$$

where x is the spatial coordinate, v is the velocity, m is the mass, and F is the force associated with a particle.

In some embodiments, loss 116 is used to calculate the error of evolution model prediction 106 in comparison to the molecular dynamics simulation result.

Monte Carlo simulation is a computational method that uses random sampling to model and analyzes complex systems or processes. In some embodiments, in a Monte Carlo simulation, a large number of random trials are run to generate statistical distributions that represent the range of possible outcomes for a given particle or system. These trials are based on a set of input parameters that are varied randomly within their defined ranges. The output of each trial is recorded, and the resulting data is used to estimate the probability distribution of outcomes. The Monte Carlo simulations are useful to analyze the behavior of particles in complex systems. As used herein, a molecular dynamics simulation encompasses Monte Carlo simulations.

In some embodiments, the particle information determined by the simulation updater {X_{MD}, t_{MD}} may be used as particle information at the next time step {Xₖ₊₁, tₓ₊₁} 118 and may be used to train the evolution model 108. For example, the particle information determined by the simulation updater 110 may be stored in a training database of the evolution model 108 as training data and may be used to train the predictions of particle data of the evolution model 108. In some embodiments, the particle information determined by the simulation updater 110 {X_{MD}, t_{MD}} may also be compared to the particle information determined by the evolution model 108 to determine an uncertainty of the evolution model U_{EV} 114.

In some embodiments, the evolution model 108 may also be trained using experimental data. For example, the experimental data may be stored in the training database of the evolution model 108 with appropriate labels to train the evolution model 108.

The global model 106, evolution model 108, and simulation updater 110 as depicted in FIG. 1 together form the training stage of the 104 of the evolution model 108.

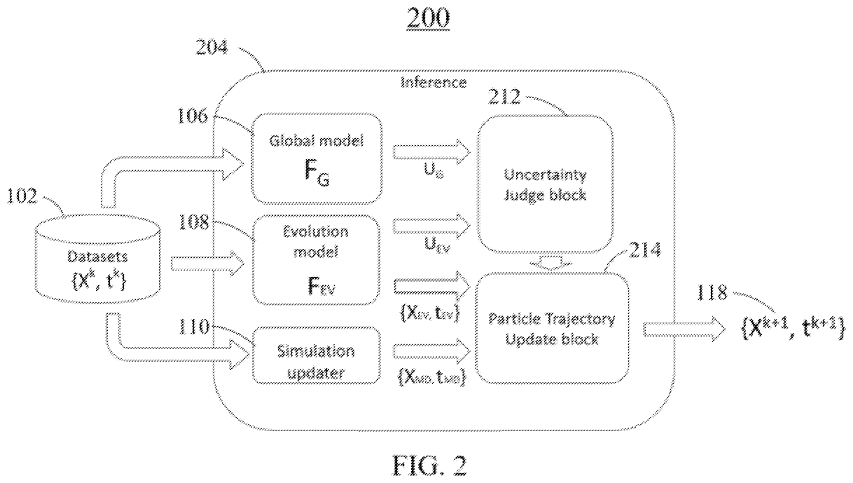

At the inference stage 204 of FIG. 2, the output of evolution and global models are directly provided with the uncertainty judge block and particle trajectory update block, and the next time-step values are calculated with appropriate updater.

FIG. 2 schematically illustrates a framework for performing inference using the trained networks, according to an embodiment of the present invention. FIG. 2 is similar to FIG. 1 as it depicts datasets 102 that include all particle data (X^{k}) at corresponding time steps (t^{K}). As shown in FIG. 1, FIG. 2 also depicts that the data from datasets 102 is provided to a global model (F_{G}) 106, an evolution model (F_{EV}) 108, and a simulation updater 110. The uncertainty outputs U_{G} and U_{EV} of the global model 106 and the evolution model 108 respectively are provided to uncertainty judge block 212. In some embodiments, the uncertainty judge block 212 outputs a Boolean (True/False) value. The Boolean value indicates whether to use the prediction from the evolution model 106 or the simulation updater 110 to determine particle information at the next time step.

According to an embodiment of the present invention, the uncertainty judge block 212 accepts the different uncertainties U_{G} (that is associated with the global model 106) and U_{EV} (that is associated with the evolution model 108) as inputs and determines which of one way of the simulation updater 110 and the evolution model 108 should be used to calculate the particle information at the next time step {X^{k+1}, t^{K+1}} using the particle information from the dataset {X^{k}, t^{K}}. In some embodiments, the uncertainty judge block 212 may be implemented by that normalizing the estimated uncertainties U_{G} and U_{EV} between 0 and unity, and the uncertainty judge block 212 may be configured to direct or use the simulation updater if both uncertainties are larger than a threshold value, e.g., 0.5. It is also possible to use a mathematical function f(U_{G}, U_{EV}) to unify the two uncertainties. In some embodiments, the uncertainty judge block 212 compares the received uncertainties U_{G} and U_{EV} to a threshold. In response to determining that the received uncertainties are less than a threshold, the uncertainty judge block 212 may instruct the particle trajectory block 214 to use the evolution model 108 to determine the particle information at the next time step. In response to determining that the received uncertainties are greater than a threshold, the uncertainty judge block 212 may instruct the particle trajectory block 214 to use the simulation updater 110 to determine the particle information at the next time step. Exemplary source code summarizing the operation of the particle trajectory block 214 is shown below:

```
if max(U_{G}, U_{EV}) > threshold:
  X^{k+1} = X_{MD}
  t^{k+1} = t_{MD}
else:
  X^{k+1} = X_{EV}
  t^{k+1} = t_{EV},
where threshold is a hyper-parameter, e.g., 0.5.
```

In some embodiments, the received uncertainties U_{G} and U_{EV} may be combined into a single uncertainty by the uncertainty judge block 212, and the combined uncertainty may be compared to a threshold In some examples, the received uncertainties U_{G} and U_{EV} may be compared to individual respective thresholds and if both uncertainties are over the respective thresholds, the uncertainty judge block 212 may instruct the particle trajectory block 214 to use the simulation updater 110 to determine the particle information at the next time step. In case both the uncertainties are less than the respective thresholds, the uncertainty judge block 212 may instruct the particle trajectory block 214 to use the evolution model 108 to determine the particle information at the next time step. In some embodiments, the greater of the measured uncertainties U_{G} and U_{EV} may be compared to a threshold to determine whether to use the simulation updater 110 or evolution model 108.

The evolution model 108 also generates an estimation of particle data X_{EV} using a machine learning model which may be provided to particle trajectory update block 214. Similarly, simulation updater 110 generates an estimation of particle data X_{MD} using molecular dynamics/Monte Carlo simulations which may be provided to particle trajectory update block 214. In some embodiments, the molecular dynamics/Monte Carlo simulations use the Newton equation to update the particle coordinate and velocity. Monte Carlo simulations are also a form of a simulation to describe particle evolution, and as used herein, are also understood to be a molecular dynamics simulation. The particle trajectory update block 214 uses either the machine learning model of the evolution model 108 or the molecular dynamics/Monte Carlo simulations of simulation updater 110 to generate particle information at the next time step {X^{k+1}, t^{k+1}} 118. In some embodiments, the decision to select either the machine learning model of the evolution model 108 or the molecular dynamics/Monte Carlo simulations of simulation updater 110 is based on the output of the uncertainty judge block 212 received at particle trajectory update block 214.

According to embodiments of the present invention, the evolution model 108 may be implemented using graph neural network-based models, such as GemNet and SchNet. Transformer-type models, such as Graphomer and Equiformer, could also be used. The listed models do not provide uncertainty of the prediction. For this purpose, probabilistic transformer may be adopted which provides the prediction uncertainty (see Franke, Jörg KH, Frederic Runge, and Frank Hutter, “Probabilistic Transformer: Modelling Ambiguities and Distributions for RNA Folding and Molecule Design,” arXiv preprint arXiv:2205, 13927 (2022), which is hereby incorporated by reference herein).

According to embodiments of the present invention, the evolution model 108 may directly compare the result of molecular dynamics simulation and an output of the machine learning model of the evolution model 108 on a sub-group of particles. For example, the difference between the output of the machine learning model and the molecular dynamics simulation may be used to determine the uncertainty U_{EV} 114 of the evolution model 108.

The global model 106 and evolution model 108 may be trained in a supervised manner using those uncertainties as a label if it is available. For example, a typical supervised model training trains the global model 106 and evolution model 108 may using the exemplary source code as shown below:

```
for e in epoch:
  for data, label_1, label_2 in dataloader:
    prediction, uncertainty_1 = model_1(data)
    uncertainty_2 = model_2(data)
    loss_1 = loss_1(prediction, label_1)
    loss_2 = loss_2(uncertainty_2, label_2)
    loss_1.backward( )
    loss_2.backward( )
```

Since the global model describes physical and chemical changes of the considering system, the uncertainty U_{G} 112 can be estimated from the observational data, such as temporal evolution of the drug and protein molecules. In the case of the interaction between a drug and a protein, the distance between them can be used for the uncertainty measure because the interaction becomes strong as the distance decreases.

On the other hand, the uncertainty U_{EV} 114 of the evolution model can be obtained using Bayesian method. One simple way is to construct the evolution model using the families of probabilistic models, such as the Gaussian process and variational autoencoder (see Pinheiro Cinelli, Lucas; et al. “Variational Autoencoder,” Variational Methods for Machine Learning with Applications to Deep Networks, Springer, pp. 111-149 (2021), which is hereby incorporated by reference herein).

In some embodiments, molecular dynamics simulations are used to simulate a process of an interaction between source and target molecules, such as drug and protein. Currently, molecular dynamics simulations are relatively accurate, but computationally very heavy, which prohibits an efficient search of new drug. Although machine learning techniques have started to be used in this area, current machine learning models assume a static target protein, and therefore suffer from the technical limitations of being able to capture dynamic nature of the target protein. The hybrid method according to embodiments of the present invention can solve this problem because the hybrid method accelerates the calculation using machine learning models which do not have any restriction on the time-step size and the numerical cost are usually cheaper than the molecular dynamics simulation. In addition, owing to the uncertainty measure, the hybrid method safely detects a sign of the global change of the target protein structure and performs its calculation using molecular dynamics simulation. In this case, the hybrid method can use either a value of the local potential energy in a target protein or the distance between drug and target protein as the global uncertainty estimation rule, for example. In some embodiments, the potential sum-up of the potential energy of all particles in a protein is calculated using assisted model building with energy refinement (AMBER). In some examples, when the distance between a drug and a target protein is a few multiples of the target protein size, the particle trajectory block 214 switches from using the simulation updater 110 instead of the evolution model 108 to determine the particle information at the next time step. For example, the particle trajectory block 214 uses the evolution model 108 to determine particle information at the next time step when the distance between the drug and the target protein is more than k*protein size, where k is a numeric value (e.g., two). In case the distance between the drug and the target protein is less than k*protein size, the particle trajectory block 214 uses the simulation updater 110. In some embodiments, if there is a well-known uncertainty measure of global estimation as a rule (e.g., distance), it can be directly used.

Similar to the embodiments described above, the hybrid method can also be applied to find a new material with a desirable property. The molecular dynamics simulation can also be used for finding the properties of a new material, such as elasticity. For example, elasticity of a substance is estimated by adding an external force on the particles that constitute the substance and observing a variation the macroscopic physical variables, such as density and pressure, typically, 100 n see/1 f sec=10⁸ steps. In some embodiments, hydrodynamic simulation could be used.

The hybrid method allows to accelerate to obtain those properties while using less processing power than the molecular dynamics simulation with similar accuracy. In this case, a value of the local potential energy or the entropy in the material molecule is used as the global uncertainty estimation rule, for example.

As used herein, the molecular dynamics simulation includes Monte Carlo simulations. In this case, the molecular dynamics updater block is substituted by a Monte Carlo updater block. The remaining part can be the same.

The hybrid method can also be used to simulate the dynamic evolution of proteins which is important to understand the properties of the proteins, such as a target protein for a newly developed drug. In this case, the hybrid method can use a value of the local potential energy in a target as the global uncertainty estimation rule, for example. An exemplary list of steps for simulating a dynamic evolution of proteins is included below:
1. Perform simulation.
2. Calculate variables relating with the protein evolution, such as the volume and surface area.
3. Identify a possible location where a drug can be attached and the structure stability around the location.

In some embodiments, similar procedures can be used to find a possibility of an adsorber and catalyst pair's adsorption process.

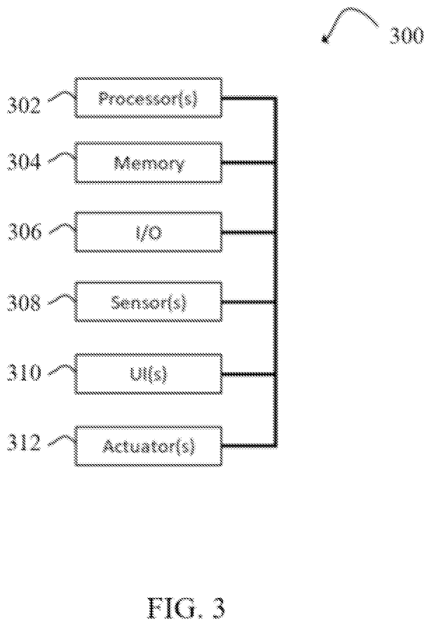

Referring to FIG. 3, a processing system 300 can include one or more processors 302, memory 304, one or more input/output devices 306, one or more sensors 308, one or more user interfaces 310, and one or more actuators 312. Processing system 300 can be representative of each computing system disclosed herein.

Processors 302 can include one or more distinct processors, each having one or more cores. Each of the distinct processors can have the same or different structure. Processors 302 can include one or more central processing units (CPUs), one or more graphics processing units (GPUs), circuitry (e.g., application specific integrated circuits (ASICs)), digital signal processors (DSPs), and the like. Processors 302 can be mounted to a common substrate or to multiple different substrates.

Processors 302 are configured to perform a certain function, method, or operation (e.g., are configured to provide for performance of a function, method, or operation) at least when one of the one or more of the distinct processors is capable of performing operations embodying the function, method, or operation. Processors 302 can perform operations embodying the function, method, or operation by, for example, executing code (e.g., interpreting scripts) stored on memory 304 and/or trafficking data through one or more ASICs. Processors 302, and thus processing system 300, can be configured to perform, automatically, any and all functions, methods, and operations disclosed herein. Therefore, processing system 300 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 300 can be configured to perform task “X”. Processing system 300 is configured to perform a function, method, or operation at least when processors 302 are configured to do the same.

Memory 304 can include volatile memory, non-volatile memory, and any other medium capable of storing data. Each of the volatile memory, non-volatile memory, and any other type of memory can include multiple different memory devices, located at multiple distinct locations and each having a different structure. Memory 304 can include remotely hosted (e.g., cloud) storage.

Examples of memory 304 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 304.

Input-output devices 306 can include any component for trafficking data such as ports, antennas (i.e., transceivers), printed conductive paths, and the like. Input-output devices 306 can enable wired communication via USB®, DisplayPort®, HDMI®, Ethernet, and the like. Input-output devices 306 can enable electronic, optical, magnetic, and holographic, communication with suitable memory 306. Input-output devices 306 can enable wireless communication via WiFi®, Bluetooth®, cellular (e.g., LTE®, CDMA®, GSM®, WiMax®, NFC®), GPS, and the like. Input-output devices 306 can include wired and/or wireless communication pathways.

Sensors 308 can capture physical measurements of environment and report the same to processors 302. User interface 310 can include displays, physical buttons, speakers, microphones, keyboards, and the like. Actuators 312 can enable processors 302 to control mechanical forces.

Processing system 300 can be distributed. For example, some components of processing system 300 can reside in a remote hosted network service (e.g., a cloud computing environment) while other components of processing system 300 can reside in a local computing system. Processing system 300 can have a modular design where certain modules include a plurality of the features/functions shown in FIG. 3. For example, I/O modules can include volatile memory and one or more processors. As another example, individual processor modules can include read-only-memory and/or local caches.

Embodiments of the present invention enable the following advantages and improvements over existing technology:
a. Use of uncertainty of the evolution model to judge if using the model prediction or molecular dynamics simulator's prediction.
b. Use of global geometric variation as an origin of the uncertainty of the evolution model prediction.
c. Increase the reliability of the simulation result because of the switching process between molecular dynamics simulation and machine learning model using uncertainty.
d. Speed up the molecular dynamics simulation.
e. Integrated numerical simulators and observation data.
f. Use of hybrid hardware, in particular graphics processing units (GPUs).

In an embodiment, the present invention provides a method for particle simulation using molecular dynamics and machine learning, the method comprising the steps of:
1. Setting an appropriate rule, “global uncertainty estimation rule”, for the global model (as described above);
2. Building a neural network architecture for the evolution model (as described in step a. above);
3. Building a neural network architecture for the global model or building a rule-based model (use of global geometric variation as an origin of the uncertainty of the evolution model prediction);
4. (Optionally) Training the neural networks for global and evolution models using either simulation or experimental data;
5. Preparing a molecular dynamics/Monte Carlo simulator;
6. Preparing an initial condition (coordinates and velocities of the particles);
7. Loading a pretrained weight of the neural networks (for example, as trained in step 4);
8. Setting a threshold of uncertainties for judging model; and
9. Performing a simulation by combining the simulation updater and the evolution models prediction using particle trajectory updater based on the decision of the uncertainty judge block which observes the evolution and global model uncertainties (use of uncertainty of the evolution model to judge if using the model prediction or molecular dynamics simulator's prediction, and use of global geometric variation as an origin of the uncertainty of the evolution model prediction).

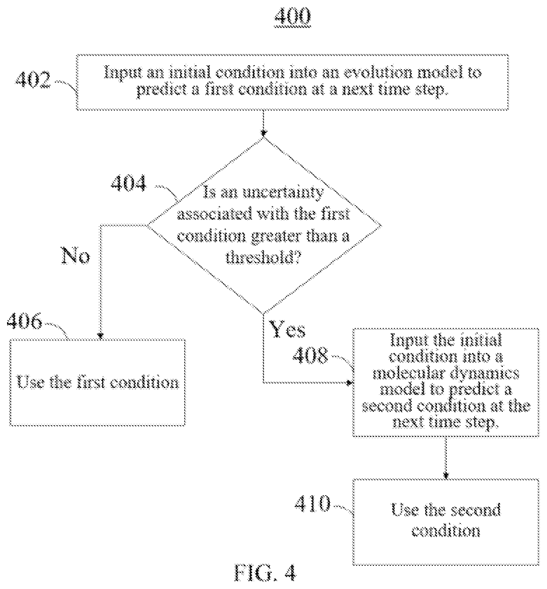

FIG. 4 illustrates an exemplary flowchart for accelerating particle simulations according to an embodiment of the present invention. In some examples, the system takes an initial condition of a particle at a first time step and determines a next condition of the particle at the next time step using the machine learning model or the molecular dynamics simulation based on a calculated uncertainty of the evolution model. It will be recognized that any of the following blocks may be performed in any suitable order and that the process 400 may be performed in any environment and by any suitable computing device and/or controller.

At step 402, an initial condition (e.g., position and velocity of a particle at an initial time step) is input into an evolution model to predict a first condition (e.g., position and velocity of the particle) at a next time step.

At step 404, a determination is made whether an uncertainty associated with the first condition is greater than a threshold. In response to determining that the uncertainty associated with the first condition is less than the threshold, the process moves to step 406 to use the first condition.

In response to determining that the uncertainty associated with the first condition is greater than the threshold, the process moves to step 408 to input the initial condition into a molecular dynamics model to predict a second condition (e.g., position and velocity of the particle) at the next time step. At step 410, the second condition is used.

Embodiments of the present invention can be advantageously applied to regression problems (continuous values) to provide improvements to various technical fields such as operation system design and optimization, material design and optimization, telecommunication network design and optimization, etc. Compared to existing approaches, embodiments of the present invention minimize uncertainty, while increasing performance and accuracy, providing for faster computation and saving computational resources and memory. For example, according to embodiments of the present invention, outliers with low uncertainty can be avoided while the latency and/or memory consumption is linear or constant.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A computer-implemented method for performing a molecular dynamics simulation, the method comprising:
inputting an initial condition into an evolution model to predict a first condition at a next time step;
inputting the initial condition into a molecular dynamics model to predict a second condition at the next time step; and
determining whether to use the first condition or the second condition as a prediction in the molecular dynamics simulation based on an estimated uncertainty associated with the evolution model.

2. The method of claim 1, wherein the initial condition includes coordinate and velocity information of a particle and corresponding surrounding particles at an initial time step, and the first and second condition each include a prediction of a position and a velocity of the particle at a next time step.

3. The method of claim 2, wherein the estimated uncertainty associated with the evolution model is based on a global uncertainty.

4. The method of claim 3, wherein the global uncertainty is associated with a global model, and wherein the global model accepts particle information and computes the global uncertainty based on a change in a global geometry of the particle.

5. The method of claim 4, wherein the global uncertainty and the uncertainty associated with the evolution model are input to an uncertainty model, and wherein the uncertainty model performs an evaluation of the global uncertainty and the uncertainty associated with the evolution model by comparing to an uncertainty threshold.

6. The method of claim 5, wherein the determination of whether to use the first condition or the second condition is based on the evaluation by the uncertainty model, and wherein the first condition is used as the prediction in the molecular dynamics simulation based on a combination of the global uncertainty and the uncertainty associated with the evolution model being lower than the uncertainty threshold.

7. The method of claim 6, wherein the uncertainty model produces a Boolean value as a result of the evaluation.

8. The method of claim 6, wherein the global model is a trained neural network, and wherein the global model is trained to estimate an uncertainty of global-geometrical changes.

9. The method of claim 8, wherein a global uncertainty estimation rule is calculated for the global model, and wherein the global uncertainty estimation rule is based on a distance calculated between the particle and a target particle in the global model or a local potential energy of the particle.

10. The method of claim 9, wherein determining whether to use the first condition or the second prediction as the prediction in the molecular dynamics simulation is based on the distance between the particle and the target particle or the local potential energy of the particle being either greater than or less than a target threshold.

11. The method of claim 10, wherein the target threshold is calculated as a numerical multiple of a size of the target particle.

12. The method of claim 1, wherein the molecular dynamics simulation is a Monte Carlo simulation.

13. The method of claim 1, wherein the evolution model is a trained neural network, and wherein the evolution model is trained using experimental and/or simulation data including the prediction used in the molecular dynamics simulation.

14. A computer system programmed for performing a molecular dynamics simulation, the computer system comprising one or more hardware processors which, alone or in combination, are configured to provide for execution of the following steps:
inputting an initial condition into an evolution model to predict a first condition at a next time step;
inputting the initial condition into a molecular dynamics model to predict a second condition at the next time step; and
determining whether to use the first condition or the second condition as a prediction in the molecular dynamics simulation based on an estimated uncertainty associated with the evolution model.

15. A tangible, non-transitory computer-readable medium for performing a molecular dynamics simulation having instructions thereon, which, upon being executed by one or more processors, provides for execution of the following steps:
inputting an initial condition into an evolution model to predict a first condition at a next time step;
inputting the initial condition into a molecular dynamics model to predict a second condition at the next time step; and
determining whether to use the first condition or the second condition as a prediction in the molecular dynamics simulation based on an estimated uncertainty associated with the evolution model.
